(19) United States
(12) Patent Application Publication
(10) Pub. No.: US 2024/0324872 A1
(43) Pub. Date: Oct. 3, 2024

(54) OPTICAL SYSTEM APPLIED TO OPTICAL BIOMETER

(71) Applicant: Crystalvue Medical Corporation, Taoyuan City 330 (TW)

(72) Inventors: Meng-Shin YEN, Taoyuan City (TW); Yen-Jen CHANG, Taoyuan City (TW); Che-Liang TSAI, Taoyuan City (TW); Chun-Nan LIN, Taoyuan City (TW); Sung-Yang WEI, Taoyuan City (TW); Hsuan-Hao CHAO, Taoyuan City (TW); Chung-Ping CHUANG, Taoyuan City (TW); William WANG, Taoyuan City (TW); Tung-Yu LEE, Taoyuan City (TW); Chung-Cheng CHOU, Taoyuan City (TW)

(21) Appl. No.: 18/620,005

(22) Filed: Mar. 28, 2024

Related U.S. Application Data
(60) Provisional application No. 63/455,621, filed on Mar. 30, 2023.

(51) Int. Cl. A61B 3/00; A61B 3/10

(52) U.S. Cl. CPC A61B 3/0075; A61B 3/1025

(57) ABSTRACT

An optical system applied to an optical biometer is disclosed. The optical system includes a light source, first and second switchable reflectors, and first and second fixed reflectors. The first switchable reflector is disposed corresponding to the light source. The second switchable reflector is disposed corresponding to an eye. In a first mode, the first and second switchable reflectors are switched to a first state, and the incident light emitted by the light source is reflected by the first fixed reflector along a first optical path and then emitted to a first position of the eye. In a second mode, the first and second switchable reflectors are switched to a second state, and the incident light is sequentially reflected by the first switchable reflector, the second fixed reflector and the second switchable reflector along a second optical path and then emitted to a second position of the eye.

### BACKGROUND OF THE INVENTION

### Cross Reference to Related Applications

This Application is a non-provisional application claiming priority to U.S. Provisional Application 63/455,621 filed on Mar. 30, 2023, which is incorporated by reference in its entirety.

### 1. FIELD OF THE INVENTION

The invention relates to a biometer; in particular, to an optical system applied to an optical biometer.

### 2. DESCRIPTION OF THE PRIOR ART

In a conventional optical coherent interferometric biometer, an incident light emitted by a light source is divided into a reference light and a sensing light and then transmitted to a reference arm and a sensing arm respectively. After the reference light and the sensing light are respectively reflected, coupled and interfered, they are detected and analyzed by the detector to obtain the relative position of each interface of the eye.

However, the conventional optical coherent interferometric biometer still has the following shortcomings:
(1) When measuring interfaces at different depths in the eye, if the optical path range of the reference arm needs to be shared, the optical path difference between the anterior chamber and the fundus of the eye needs to be compensated at the sensing arm. However, the sensing arm design used in the conventional optical coherent interferometric biometer cannot effectively compensate for this optical path difference. In addition, the conventional optical coherent interferometric biometer cannot quickly switch among different focus depths. Therefore, the interface for measuring different depths in the eye cannot have optimal signals at all different depths, which affects the accuracy of the measurement and needs to be further improved.
(2) When measuring interfaces at different depths in the eye, if the optical path lengths of the sensing arms are equal, the reference arm needs to have a larger optical path modulation range to cover different optical path lengths required by both the anterior chamber and the fundus of the eye, and the reference arm also needs to be able to quickly switch among different optical path lengths. However, the reference arm design used in the conventional optical coherent interferometric biometer cannot meet the above requirements and needs to be further improved.

### SUMMARY OF THE INVENTION

Therefore, the invention provides an optical system applied to an optical biometer to solve the above-mentioned problems of the prior arts.

A preferred embodiment of the invention is an optical system applied to an optical biometer. In this embodiment, the optical system includes a light source, a first switchable reflector, a second switchable reflector, a first fixed reflector and a second fixed reflector. The light source is configured to emit an incident light. The first switchable reflector is disposed corresponding to the light source and selectively switched to a first state or a second state. The second switchable reflector is disposed corresponding to an eye and selectively switched to the first state or the second state. The first fixed reflector is disposed corresponding to the first switchable reflector and the second switchable reflector. The second fixed reflector is disposed corresponding to the first switchable reflector, the second switchable reflector and the first fixed reflector. In a first mode, the first switchable reflector and the second switchable reflector are switched to the first state, and the incident light is emitted to the first fixed reflector and reflected by the first fixed reflector along a first optical path and then emitted to a first position of the eye. In a second mode, the first switchable reflector and the second switchable reflector are switched to the second state, and the incident light is sequentially reflected by the first switchable reflector, the second fixed reflector and the second switchable reflector along a second optical path and then emitted to a second position of the eye.

In an embodiment, the first position of the eye is retina and the first mode is retina mode. In an embodiment, the second position of the eye is cornea and the second mode is corneal mode.

In an embodiment, in the first state, the first switchable reflector is not positioned between the light source and the first fixed reflector and the second switchable reflector is not positioned between the first fixed reflector and the eye.

In an embodiment, in the second state, the first switchable reflector is positioned between the light source and the first fixed reflector and the second switchable reflector is positioned between the first fixed reflector and the eye.

In an embodiment, the optical system further includes a transmission mechanism, coupled to the first switchable reflector and the second switchable reflector and configured to control position switching of the first switchable reflector and the second switchable reflector.

In an embodiment, the optical biometer is an optical coherent interference biometer including: an optical splitter, configured to divide the incident light emitted by the light source into a reference light and a sensing light; a reference arm, configured to reflect the reference light to generate a first reflected light; a sensing arm, configured to emit the sensing light to the eye and receive a second reflected light from the eye; and a sensor, configured to receive the first reflected light and the second reflected light respectively and generate a sensing result.

In an embodiment, the sensing arm shares the optical path of the reference arm; the sensing arm includes a lens barrel, a first set of lenses and a second set of lenses; the sensing arm uses the lens barrel to allow the sensing light to be directed to the eye through the first set of lenses or the second set of lenses along different optical paths, so the sensing light is focused at different depths in the eye.

In an embodiment, the sensing arm includes a switching mechanism, a first set of lenses and a second set of lenses; the sensing light is emitted to the eye along the same optical path; the sensing arm switches the first set of lenses or the second set of lenses positioned on the optical path through the switching mechanism, so the sensing light is focused at different depths in the eye.

In an embodiment, the sensing arm includes a first set of lenses and a second set of lenses; the sensing light is emitted to the eye along the same optical path; the sensing arm moves the first set of lenses or the second set of lenses positioned, so the first set of lenses or the second set of lenses positioned is positioned on the optical path.

In an embodiment, the sensing arm shares the optical path of the reference arm; the sensing arm includes a lens barrel, a first set of lenses, a second set of lenses and a third set of lenses; the sensing arm allows the sensing light to pass through the first set of lenses, the second set of lenses or the third set of lenses along different optical paths to the eye by changing the state of the lens barrel.

In an embodiment, when the sensing light is emitted to the eye through the first set of lenses, the sensing light is focused on a fundus of the eye; when the sensing light is emitted to the eye through the second set of lenses, the sensing light is focused on a crystalline lens of the eye; when the sensing light is emitted to the eye through the third set of lenses, the sensing light is focused on a cornea of the eye.

In an embodiment, the lens barrel is also combined with a lens, when the lens barrel rotates, the first set of lenses, the second set of lenses or the third set of lenses is switched, so the sensing light is emitted to the eye along different optical paths to be focused on different depths in the eye.

In an embodiment, the sensing arm shares the optical path of the reference arm. The sensing arm includes a lens barrel, a first set of lenses, a second set of lenses and a third set of lenses. A mirror in the lens barrel is provided with a hole at a specific eccentricity. When the lens barrel rotates to a specific angle, the sensing light passes through the hole and then passes through the first set of lenses, the second set of lenses or the third set of lenses and emitted to the eye, so the sensing light is focused at different depths in the eye.

In an embodiment, the reference arm includes a mirror set turntable rotating to quickly switch different mirrors corresponding to different optical path lengths to quickly switch between different optical path lengths.

In an embodiment, the mirror set turntable is used with a short distance moving platform to scan back and forth in a short distance.

In an embodiment, the reference arm is linked with the sensing arm through a linkage mechanism to increase a measurement speed.

In an embodiment, the reference arm includes a plurality of different mirror set turntables provided with holes at certain angles, so the reference light passes through the holes and a front mirror set turntable and is then reflected by a mirror of a rear mirror set turntable to increase the flexibility of optical path control.

Compared to the prior art, the optical system applied to the optical biometers proposed by the invention has the following advantages and effects:
(1) When the optical biometer of the invention measures interfaces at different depths in the eye, if it is necessary to share the optical path range of the reference arm, the sensing arm design used in the optical system can effectively compensate the optical path difference between the anterior chamber and the fundus and can quickly switch among different focus depths, so it can have the best signal when measuring interfaces at different depths in the eye to improve measurement accuracy.
(2) When the optical biometer of the invention measures interfaces at different depths in the eye, if the optical path lengths of the sensing arms are equal, the reference arm used in the optical system has a large optical path modulation range and can quickly switch among different optical path lengths required for interfaces at different depths in the eye, so it can effectively improve the shortcomings of the prior arts.

The advantage and spirit of the invention may be understood by the following detailed descriptions together with the appended drawings.

### BRIEF DESCRIPTION OF THE APPENDED DRAWINGS

FIG. 1 illustrates a schematic diagram of an optical system applied to an optical biometer according to a preferred embodiment of the invention.

FIG. 2 illustrates a schematic diagram of an optical system applied to an optical coherent interferometric biometer in the invention.

FIG. 3A to FIG. 9 illustrate schematic diagrams of different embodiments of a sensing arm in an optical system of the invention respectively.

FIG. 10 illustrates a schematic diagram of an embodiment of a reference arm in an optical system of the invention.

### DETAILED DESCRIPTION OF THE INVENTION

Reference will now be made in detail to the exemplary embodiments of the invention, examples of which are illustrated in the accompanying drawings. Elements/components with the same or similar numbers used in the drawings and embodiments are intended to represent the same or similar parts.

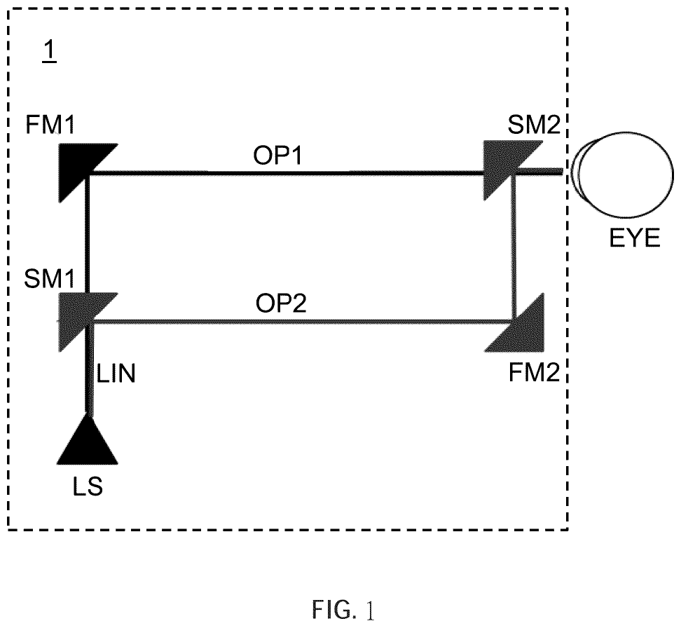

A specific embodiment of the invention is an optical system applied to an optical biometer. In this embodiment, the optical biometer is used to detect and measure the eyes, but not limited to this. Please refer to FIG. 1. FIG. 1 illustrates a schematic diagram of an optical system applied to an optical biometer in this embodiment.

As shown in FIG. 1, the optical system 1 applied to the optical biometer includes a light source LS, a first switchable reflector SM1, a second switchable reflector SM2, a first fixed reflector FM1 and a second fixed reflector FM2. The first switchable reflector SM1 is disposed corresponding to the light source LS and is selectively switched to a first state or a second state. The second switchable reflector SM2 is disposed corresponding to the eye EYE and is selectively switched to the first state or the second state. The first fixed reflector FM1 is disposed corresponding to the first switchable reflector SM1 and the second switchable reflector SM2. The second fixed reflector FM2 is disposed corresponding to the first switchable reflector SM1, the second switchable reflector SM2 and the first fixed reflector FM1. The light source LS is used to emit an incident light LIN.

In a first mode, the first switchable reflector SM1 and the second switchable reflector SM2 are switched to the first state, and the incident light LIN emitted by the light source LS is emitted to the first fixed reflector FM1 and reflected by the first fixed reflector FM1 along a first optical path OP1 and then transmitted to a first position of the eye EYE. In a second mode, the first switchable reflector SM1 and the second switchable reflector SM2 are switched to the second state, and the incident light LIN to be sequentially reflected by the first switchable reflector SM1, the second fixed reflectors FM2 and the second switchable reflector SM2 along a second optical path OP2 and then transmitted to a second position of the eye EYE.

It should be noted that in the first state, the first switchable reflector SM1 is not positioned between the light source LS and the first fixed reflector FM1 and the second switchable reflector SM2 is not positioned between the first fixed reflector FM1 and the eye EYE, so that the incident light LIN can be directed to the first fixed reflector FM1 along the first optical path OP1 and then reflected by the first fixed reflector FM1 to the eye EYE; in the second state, the first switchable reflector SM1 is positioned between the light source LS and the first fixed reflector FM1 and the second switchable reflector SM2 is positioned between the first fixed reflector FM1 and the eye EYE, so that the incident light LIN can be sequentially reflected by the first switchable reflector SM1, the second fixed reflector FM2 and the second switchable reflector SM2 along the second optical path OP2 and then be directed to the eye EYE.

In practical applications, the first position and the second position of the eye EYE can be set as the retina and cornea located at different depths in the eye EYE respectively, and the first mode and the second mode can be set as the retinal mode and corneal mode respectively, but not limited to this. The optical system can further include a transmission mechanism coupled to the first switchable reflector SM1 and the second switchable reflector SM2 for controlling position switching of the first switchable reflector SM1 and the second switchable reflector SM2, but not limited to this.

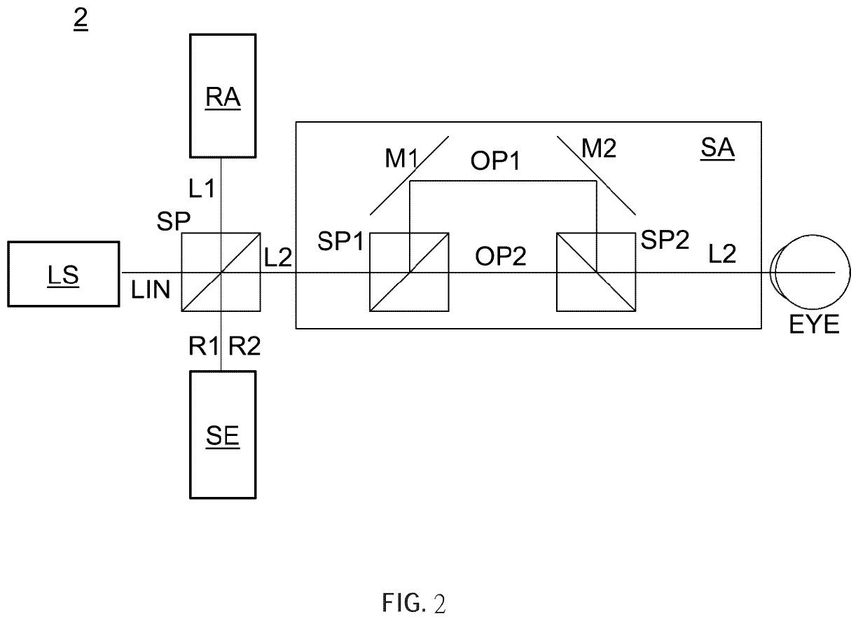

Please refer to FIG. 2. FIG. 2 illustrates a schematic diagram of the optical system applied to an optical coherent interference biometer in the invention. As shown in FIG. 2, the optical coherent interferometric biometer 2 includes a light source LS, an optical splitter SP, a reference arm RA, a sensing arm SA and a sensor SE. The optical splitter SP is used to divide an incident light LIN emitted by the light source LS into a reference light L1 and a sensing light L2, and then emit the reference light L1 and the sensing light L2 to the reference arm RA and the sensing arm SA respectively. The reference arm RA is used to reflect the reference light L1 to form a first reflected light R1. The sensing arm SA is used to transmit the sensing light L2 to an eye EYE and receive a second reflected light R2 from the eye EYE. The sensor SE is used to receive the first reflected light R1 and the second reflected light R2 respectively and generate a sensing result according to the first reflected light R1 and the second reflected light R2.

It should be noted that the design of the sensing arm used in the conventional optical coherent interferometric biometer has poor compensation effect on an optical path difference between an anterior chamber and a fundus of the eye caused by sharing the optical path range of the reference arm. Therefore, in order to improve the compensation effect of the sensing arm for the optical path difference, in this embodiment, the sensing arm SA of the invention can include a first optical splitter SP1, a second optical splitter SP2, a first mirror M1 and a second mirror M2. And, the sensing light L2 can be directed to different positions of the eye EYE through the design of the first beam splitter SP1, the second beam splitter SP2, the first mirror M1 and the second mirror M2, thereby the optical path difference is generated.

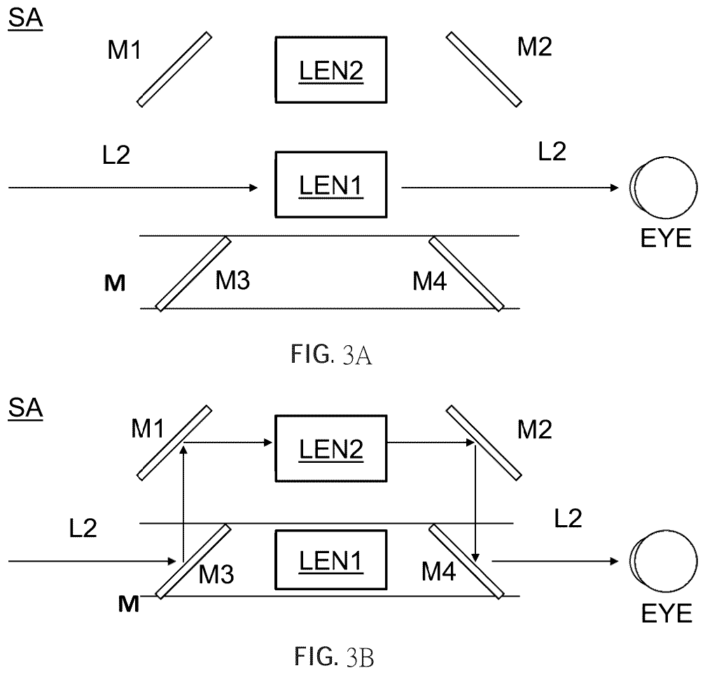

In addition to the above embodiments, the invention also proposes the following other different designs for the sensing arm SA in the optical system 1 to effectively compensate for the optical path difference between the anterior chamber and the fundus of the eye EYE caused by sharing the optical path range of the reference arm RA. Next, different embodiments shown in FIG. 3A to FIG. 9 will be described in detail.

Please refer to FIG. 3A and FIG. 3B. The design of the sensing arm SA shown in FIG. 3A and FIG. 3B is suitable for the range of the optical path of the common reference arm RA. The sensing arm SA includes a lens barrel M, a first mirror M1, a second mirror M2, a first set of lenses LEN1 and a second set of lenses LEN2. A third mirror M3 and a fourth mirror M4 are disposed in the lens barrel M. The sensing arm SA can change the position of the lens barrel M to allow the sensing light L2 to be directed to the eye EYE through the first set of lenses LEN1 or the second set of lenses LEN2 along different optical paths, so that the sensing light L2 can be focused on different depths in the eye EYE to obtain the best signal.

For example, as shown in FIG. 3A, when the position of the lens barrel M is located below the first set of lenses LEN1, the sensing light L2 emitted to the first set of lenses LEN1 can be directly emitted to the eye EYE through the first set of lenses LEN1. As shown in FIG. 3B, when the position of the lens barrel M moves upward, the third mirror M3 and the fourth mirror M4 in the lens barrel M are positioned on both sides of the first set of lenses LEN1, the sensing light L2 is reflected by the third mirror M3 and the first mirror M1 in sequence and passes through the second set of lenses LEN2, and then is reflected by the second mirror M2 and the fourth mirror M4 in sequence and emitted to the eye EYE. Thereby, the design of the sensing arm SA shown in FIG. 3A and FIG. 3B can focus the sensing light L2 on different depths in the eye EYE to obtain the best signal.

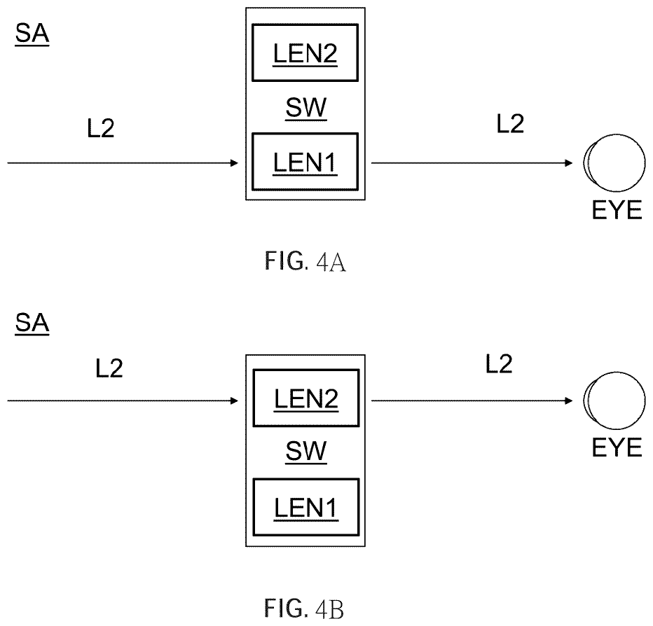

Please refer to FIG. 4A and FIG. 4B. The design of the sensing arm SA shown in FIG. 4A and FIG. 4B is suitable for a range in which the optical path of the reference arm RA can cover the anterior chamber and the fundus of the eye EYE. The sensing arm SA includes a switching mechanism SW, a first set of lenses LEN1 and a second set of lenses LEN2. As shown in FIG. 4A and FIG. 4B, the sensing light L2 is emitted to the eye EYE along the same optical path, and the switching mechanism SW of the sensing arm SA is used to switch the first set of lenses LEN1 or the second set of lenses LEN2 to position on the optical path that the sensing light L2 is emitted to the eye EYE, so that the sensing light L2 can be emitted to the eye EYE through different set of lenses to focus on different depths in the eye EYE, but the transmission path and optical path of the sensing light L2 are not changed.

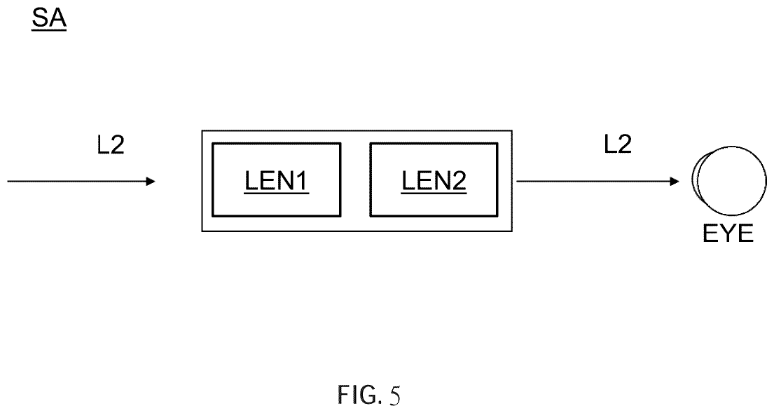

Please refer to FIG. 5. The design of the sensing arm SA shown in FIG. 5 is suitable for the reference arm RA. The optical path can cover the range of the anterior chamber and the fundus of the eye EYE. By moving different set of lenses (the relative positions of the first set of lenses LEN1 and the second set of lenses LEN2), the sensing light L2 can be focused at different depths in the eye EYE to obtain the best signal, but the transmission path and the optical path of the sensing light L2 are not changed.

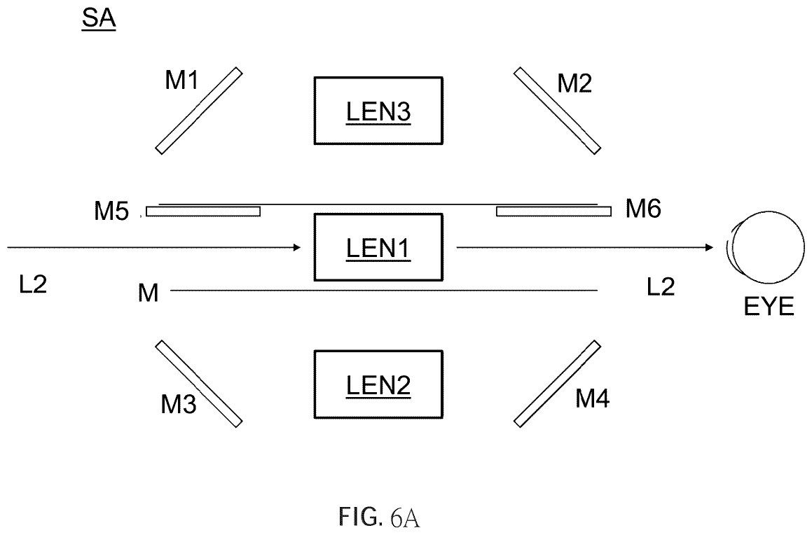

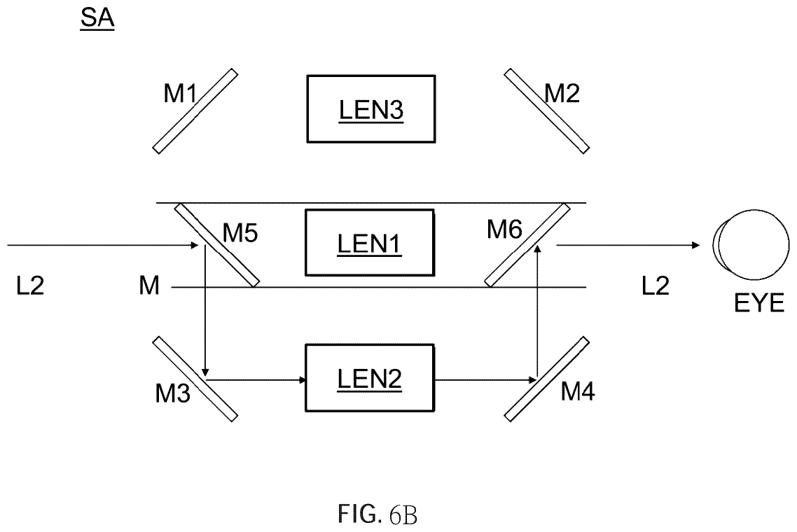

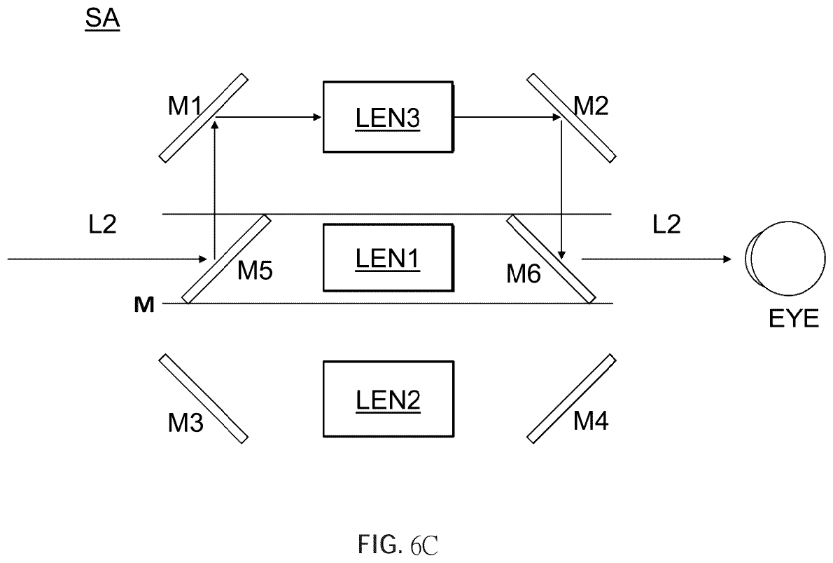

Please refer to FIG. 6A to FIG. 6C. The design of the sensing arm SA shown in FIG. 6A to FIG. 6C is suitable for the range of the optical path of the common reference arm RA. The sensing arm SA includes a lens barrel M, a first mirror M1, a second mirror M2, a third mirror M3, a fourth mirror M4, a first set of lenses LEN1, a second set of lenses LEN2 and a third set of lenses LEN3. A fifth mirror M5 and a sixth mirror M6 are disposed in the lens barrel M. By change the state of the lens barrel M, the sensing arm SA can allow the sensing light L2 to emit to the eye EYE along different optical paths through the first set of lenses LEN1, the second set of lenses LEN2 or the third set of lenses LEN3 respectively, so that the sensing light L2 can be focused on different depths in the eye EYE.

For example, as shown in FIG. 6A, when the lens barrel M rotates and changes to the first state, the fifth mirror M5 and the sixth mirror M6 in the lens barrel M are retracted upward and flat against the barrel wall, so that the sensing light L2 directed to the first set of lenses LEN1 can directly pass through the first set of lenses LEN1 and focus on the fundus of the eye EYE, but not limited to this.

As shown in FIG. 6B, when the lens barrel M rotates and changes to the second state, the fifth mirror M5 and the sixth mirror M6 in the lens barrel M are placed obliquely and parallel to the third mirror M3 and the fourth mirror M4 below them respectively, so that the sensing light L2 is sequentially reflected by the fifth mirror M5 and the third mirror M3 and passes through the second set of lenses LEN2, and then is sequentially reflected by the fourth mirror M4 and the sixth mirror M6 and focuses on the crystalline lens of the eye EYE, but not limited to this.

As shown in FIG. 6C, when the lens barrel M rotates and changes to the third state, the fifth mirror M5 and the sixth mirror M6 in the lens barrel M are placed obliquely and parallel to the first mirror M1 and the second mirror M2 above them respectively, so that the sensing light L2 is sequentially reflected by the fifth mirror M5 and the first mirror M1 and passes through the third set of lenses LEN3, and then is sequentially reflected by the second mirror M2 and the sixth mirror M6 and focuses on the crystalline lens of the eye EYE, but not limited to this.

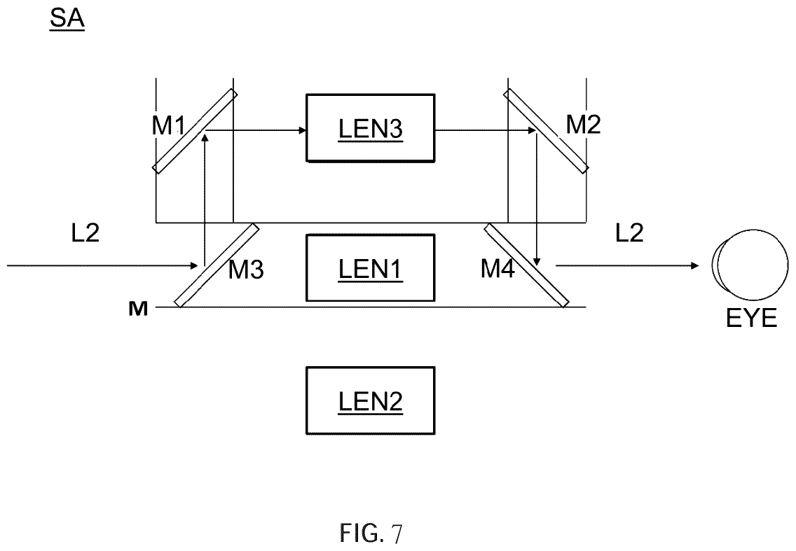

Please refer to FIG. 7. The design of the sensing arm SA shown in FIG. 7 is suitable for the range of the optical path of the common reference arm RA. The difference between the sensing arm SA in FIG. 7 and the previous embodiment is that the sensing arm SA in FIG. 7 combines the first mirror M1 and the third mirror M3 with the lens barrel M. When the lens barrel M rotates, different sets of lenses (the first set of lenses LEN1 to the third sets of lenses LEN3) can be switched to allow the sensing light L2 to pass through, so the number of mirrors used can effectively reduce. However, media of different thicknesses need to be placed in the set of lenses to achieve the optical path adjusting effect.

In practical applications, if the sensing arm SA of FIG. 7 is used with the reference arm RA with a large optical path, there is no need to insert media in the set of lenses to adjust the optical path. In addition, if there are focusing requirements of more layer structures or more different depths, more sets of lenses can be used without increasing the complexity of the mechanism. For a small amount but a need for quick switching, in addition to accelerating the rotation speed of the lens barrel M, the same set of lenses can also be duplicated and placed staggered, but not limited to this.

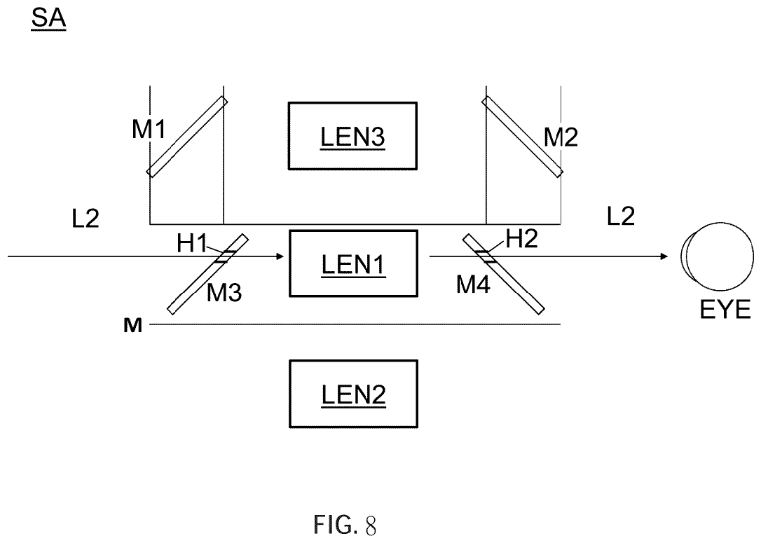

Please refer to FIG. 8. The design of the sensing arm SA shown in FIG. 8 is suitable for the range of the optical path of the common reference arm RA. The difference between the sensing arm SA in FIG. 8 and the previous embodiment is that the third mirror M3 in the lens barrel M is provided with a first hole H1 at certain eccentricities and the fourth mirror M4 is provided a second hole H2 at certain eccentricities, and the positions of the first hole H1 and the second hole H2 will correspond to each other. When the lens barrel M rotates to a specific angle, the sensing light L2 can sequentially pass through the first hole H1 on the third mirror M3, the first set of lenses LEN1 and the second hole H2 on the fourth mirror M4 before being emitted to the eye EYE. When the lens barrel M rotates to other angles, the sensing light L2 is reflected upward or downward by the third mirror M3 and emitted to the eye EYE through the second set of lenses LEN2 or the third set of lenses LEN3, so that the sensing light L2 can focus on different depths in the eye EYE.

It should be noted that the advantage of this embodiment is that it can increase the frequency of the sensing light L2 passing through the first set of lenses LEN1, thereby increasing the frequency of measuring a specific position of the eye EYE. For example, since the cornea is often used as a reference for position measurement, during the process of measuring interfaces at different depths, if the position of the cornea can be continuously measured repeatedly, it will enhance to the stability and accuracy of the measurement.

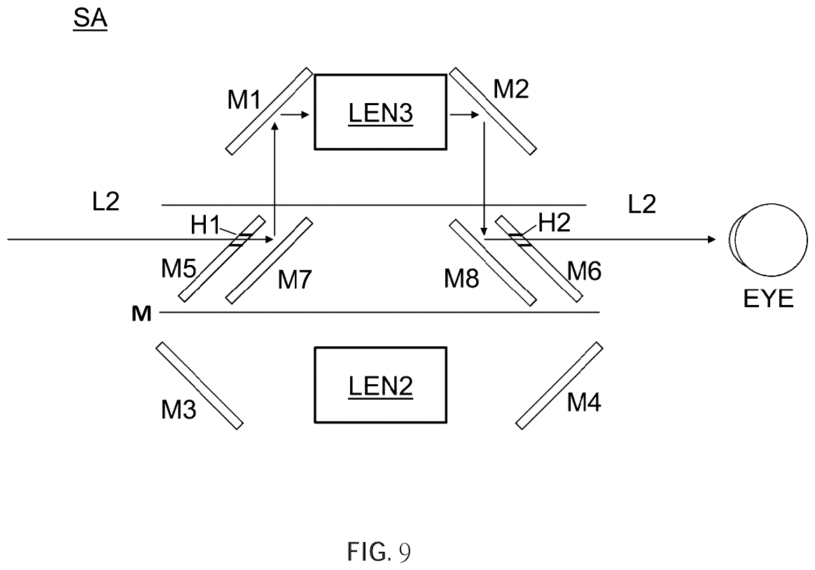

Please refer to FIG. 9. In another embodiment, the lens barrel M includes a fifth mirror M5 provided with a first hole H1 at certain eccentric places and a fifth mirror M5 provided with a second hole H2 at certain eccentric places. In addition to the sixth mirror M6, the lens barrel M also includes a seventh mirror M7 and an eighth mirror M8. When the lens barrel M rotates to a specific angle, the sensing light L2 passes through the first hole H1 on the fifth mirror M5 and then reflected by the seventh mirror M7 and the first mirror M1 in sequence, and then pass through the third set of lens LEN3 and reflected by the second mirror M2 and the eighth mirror M8 in sequence, and pass through the second hole H2 on the sixth mirror M6 and then emitted to the eye EYE, so that it can take into account the needs of different optical path lengths when passing through different sets of lenses.

It should be noted that if a frequency domain optical coherent interference (OCT) biometer is used in the invention, the optical path of the reference arm RA in the optical system does not need to change continuously (because the structure having a certain depth range (˜3 mm) can be seen at one time, its precision does not need to be too high) and needs to be able to quickly switch between different optical paths. For example, if the apex of the cornea is used as the optical path reference, then the optical path length for measuring the front surface of the lens needs to be increased by about 3.5 mm, the optical path length for measuring the back surface of the lens needs to be increased by 5˜7 mm, and the light path of the fundus of the eye needs to be measured. The distance needs to be relatively increased by about 18˜40 mm. In addition, for biometers, the distance between different depth interfaces of the eye EYE relative to the corneal apex surface is very important. Therefore, if the cornea apex surface can be continuously and repeatedly observed when measuring the different depth interfaces of the eye EYE, it can effectively ensure the accuracy of measurement.

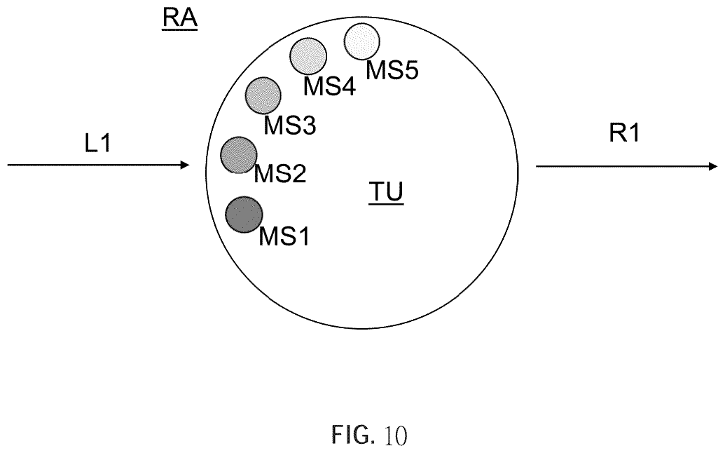

Please refer to FIG. 10, which is a schematic diagram of an embodiment of the reference arm in the optical system of the invention. In this embodiment, the reference arm RA includes a mirror set turntable TU, on which the mirrors MS1˜MS5 are disposed, and the mirrors MS1˜MS5 are placed at different depths to respectively correspond to different optical path lengths. The largest body of the mirror set turntable TU is also a mirror. The mirror set turntable TU quickly switches different mirrors MS1˜MS5 through rotation to reflect the reference light L1 incident on the reference arm RA into a reflected light R1 and then emit the reflected light R1, thereby quickly switching different optical path lengths.

It should be noted that the design of the reference arm RA can be specifically used to target the optical path of the corneal apex (or other interfaces of interest and requiring frequent measurement), so the position of the corneal apex can be frequently measured and confirmed to improve the accuracy of measuring the depth of different interfaces of the eye EYE. In addition, different mirrors can also be combined and placed repeatedly, which not only speeds up the rotation speed, but also increases the sampling frequency.

In practical applications, the mirror set turntable TU of the reference arm RA can be used with a macro moving platform such as a reciprocating screw or a piezoelectric platform to perform short-distance back-and-forth scanning. When the optical path difference between the different interface depths in the eye EYE has been roughly compensated by the sensing arm SA, the depth difference between the different mirrors of the reference arm RA can be finer and the jump and change should not be too large. When the optical path difference between different interface depths in the eye EYE is not compensated by the sensing arm SA, the depth difference between the different mirrors corresponding to the anterior chamber and the fundus of the eye EYE is large. At this time, mirror set turntables can be used to achieve fast and large optical path switching. If the sensing lights L2 of different depths can exist and be measured at the same time, the mirror of the reference arm RA corresponding to the cornea can be reduced in size, so that the reference light L1 can cover the reference mirrors and the cornea mirror of other depths, so that the signal from the cornea can continue to exist as a reference position.

In one embodiment, the reference arm RA can be linked with the sensing arm SA through a linkage mechanism such as a gear to increase the measurement speed without using expensive light reflection switching components such as galvo or micro-electromechanical systems, so that the costs can be effectively reduced.

In another embodiment, the reference arm RA can include a plurality of different mirror set turntables TU, and it can be provided with holes at certain angles so that the reference light L1 can pass through the holes and the front mirror set turntable, and then the reference light L1 is reflected by the mirror of the rear mirror set turntable to increase the flexibility and speed of the optical path control.

Compared to the prior art, the optical system applied to the optical biometers proposed by the invention has the following advantages and effects:
(1) When the optical biometer of the invention measures interfaces at different depths in the eye, if it is necessary to share the optical path range of the reference arm, the sensing arm design used in the optical system can effectively compensate the optical path difference between the anterior chamber and the fundus and can quickly switch among different focus depths, so it can have the best signal when measuring interfaces at different depths in the eye to improve measurement accuracy.
(2) When the optical biometer of the invention measures interfaces at different depths in the eye, if the optical path lengths of the sensing arms are equal, the reference arm used in the optical system has a large optical path modulation range and can quickly switch among different optical path lengths required for interfaces at different depths in the eye, so it can effectively improve the shortcomings of the prior arts.

With the example and explanations above, the features and spirits of the invention will be hopefully well described. Those skilled in the art will readily observe that numerous modifications and alterations of the device may be made while retaining the teaching of the invention.

Accordingly, the above disclosure should be construed as limited only by the metes and bounds of the appended claims.

What is claimed is:

1. An optical system applied to an optical biometer, the optical system comprising:
a light source, configured to emit an incident light;
a first switchable reflector, disposed corresponding to the light source and selectively switched to a first state or a second state;
a second switchable reflector, disposed corresponding to an eye and selectively switched to the first state or the second state;
a first fixed reflector, disposed corresponding to the first switchable reflector and the second switchable reflector; and
a second fixed reflector, disposed corresponding to the first switchable reflector, the second switchable reflector and the first fixed reflector;
wherein in a first mode, the first switchable reflector and the second switchable reflector are switched to the first state, and the incident light is emitted to the first fixed reflector and reflected by the first fixed reflector along a first optical path and then emitted to a first position of the eye; in a second mode, the first switchable reflector and the second switchable reflector are switched to the second state, and the incident light is sequentially reflected by the first switchable reflector, the second fixed reflector and the second switchable reflector along a second optical path and then emitted to a second position of the eye.

2. The optical system of claim 1, wherein the first position of the eye is retina and the first mode is retina mode.

3. The optical system of claim 1, wherein the second position of the eye is cornea and the second mode is corneal mode.

4. The optical system of claim 1, wherein in the first state, the first switchable reflector is not positioned between the light source and the first fixed reflector and the second switchable reflector is not positioned between the first fixed reflector and the eye.

5. The optical system of claim 1, wherein in the second state, the first switchable reflector is positioned between the light source and the first fixed reflector and the second switchable reflector is positioned between the first fixed reflector and the eye.

6. The optical system of claim 1, further comprising:
a transmission mechanism, coupled to the first switchable reflector and the second switchable reflector and configured to control position switching of the first switchable reflector and the second switchable reflector.

7. The optical system of claim 1, wherein the optical biometer is an optical coherent interference biometer, comprising:
an optical splitter, configured to divide the incident light emitted by the light source into a reference light and a sensing light;
a reference arm, configured to reflect the reference light to generate a first reflected light;
a sensing arm, configured to emit the sensing light to the eye and receive a second reflected light from the eye; and
a sensor, configured to receive the first reflected light and the second reflected light respectively and generate a sensing result.

8. The optical system of claim 1, wherein the sensing arm shares the optical path of the reference arm; the sensing arm comprises a lens barrel, a first set of lenses and a second set of lenses; the sensing arm uses the lens barrel to allow the sensing light to be directed to the eye through the first set of lenses or the second set of lenses along different optical paths, so the sensing light is focused at different depths in the eye.

9. The optical system of claim 1, wherein the sensing arm comprises a switching mechanism, a first set of lenses and a second set of lenses; the sensing light is emitted to the eye along the same optical path; the sensing arm switches the first set of lenses or the second set of lenses positioned on the optical path through the switching mechanism, so the sensing light is focused at different depths in the eye.

10. The optical system of claim 1, wherein the sensing arm comprises a first set of lenses and a second set of lenses; the sensing light is emitted to the eye along the same optical path; the sensing arm moves the first set of lenses or the second set of lenses positioned, so the first set of lenses or the second set of lenses positioned is positioned on the optical path.

11. The optical system of claim 1, wherein the sensing arm shares the optical path of the reference arm; the sensing arm comprises a lens barrel, a first set of lenses, a second set of lenses and a third set of lenses; the sensing arm allows the sensing light to pass through the first set of lenses, the second set of lenses or the third set of lenses along different optical paths to the eye by changing the state of the lens barrel.

12. The optical system of claim 11, wherein when the sensing light is emitted to the eye through the first set of lenses, the sensing light is focused on a fundus of the eye; when the sensing light is emitted to the eye through the second set of lenses, the sensing light is focused on a crystalline lens of the eye; when the sensing light is emitted to the eye through the third set of lenses, the sensing light is focused on a cornea of the eye.

13. The optical system of claim 11, wherein the lens barrel is also combined with a lens, when the lens barrel rotates, the first set of lenses, the second set of lenses or the third set of lenses is switched, so the sensing light is emitted to the eye along different optical paths to be focused on different depths in the eye.

14. The optical system of claim 1, wherein the sensing arm shares the optical path of the reference arm; the sensing arm comprises a lens barrel, a first set of lenses, a second set of lenses and a third set of lenses; a mirror in the lens barrel is provided with a hole at a specific eccentricity; when the lens barrel rotates to a specific angle, the sensing light passes through the hole and then passes through the first set of lenses, the second set of lenses or the third set of lenses and emitted to the eye, so the sensing light is focused at different depths in the eye.

15. The optical system of claim 1, wherein the reference arm comprises a mirror set turntable rotating to quickly switch different mirrors corresponding to different optical path lengths to quickly switch between different optical path lengths.

16. The optical system of claim 15, wherein the mirror set turntable is used with a short distance moving platform to scan back and forth in a short distance.

17. The optical system of claim 15, wherein the reference arm is linked with the sensing arm through a linkage mechanism to increase a measurement speed.

18. The optical system of claim 1, wherein the reference arm comprises a plurality of different mirror set turntables provided with holes at certain angles, so the reference light passes through the holes and a front mirror set turntable and is then reflected by a mirror of a rear mirror set turntable to increase the flexibility of optical path control.
